(19) United States
(12) Patent Application Publication
(10) Pub. No.: US 2005/0266398 A1
(43) Pub. Date: Dec. 1, 2005

(54) Method and device for rapid detection and quantitation of macro and micro matrices

(76) Inventors: Peter Lea, Toronto (CA); Shi-Fa Ding, Toronto (CA)

Correspondence Address: DIMOCK STRATTON LLP, 20 QUEEN STREET WEST SUITE 3202, BOX 102, TORONTO, ON, M5H 3R3, CA

(21) Appl. No.: 10/856,785

(22) Filed: Jun. 1, 2004

(51) Int. Cl. C12Q 1/70; G01N 33/554; G01N 33/569; C12M 1/34

(52) U.S. Cl. 435/5; 435/7.32; 435/287.2

(57) ABSTRACT

The present invention provides a method and device for rapidly detecting the presence of analytes in a sample. Quantitative and qualitative measurements of analyte concentration in a sample may be rapidly obtained. A sample including the analyte and analyte metabolites produced by the analyte are introduced into a vessel that contains a reagent or reagents that have a detectable marker and rapidly bind to the analyte and to the metabolite. The sample is then introduced to an assay device that has a loading area, a separation and a reading area. The sample is introduced into the loading area of the assay device and moves to the reading area preferably by capillary action. The methodology permits for the detection of analytes and metabolites using means for the detecting the detectable marker. The sample may be subjected to a force application means for the controlled progressive fragmentation of any analyte, which is preferably a pathogen present in the sample, into a plurality of fragments. The sample is then introduced into a vessel that contains reagents having a detectable marker that rapidly bind to the fragments of the analyte(s) to which the assay is directed. The sample is then introduced to the assay device for detection of analyte fragments. An assay device having a test dot is printed on the reading portion. The test dot includes a bound reagent that is adapted to bind to analyte fragments of the analyte for which the assay is directed. Once the fragments are bound to the test dot, the presence of the analyte fragments in the test dot can be determined by methods known in the art. The test dot may alternatively include a bound reagent that is adapted to bind to analyte or other metabolites that are produced by an analyte which is a bacterium or other pathogen to which the test is directed. The reading portion may also have a section for gathering analyte labeled with detectable markers for visual detection.

### FIELD OF THE INVENTION

The present invention includes a method for the rapid detection of analytes in a sample and a modular assay device for carrying out the method.

### BACKGROUND OF THE INVENTION

Micro and macro matrices of bacteria and their respective toxic proteinaceous contaminants account for several million cases of food-related illness and about 9,000 deaths per year in the United States. Contaminated processed food, poultry and meat products etc. are a major cause of these deaths and illnesses. The five most common pathogens infecting food products and especially poultry and meat products are E. coli O157:H7, Salmonella species, Listeria species, Listeria monocytogenes and Campylobacter jejuni.

Assays for detecting these and other microorganisms require that the samples be cultured. A culture refers to a particular strain or kind of organism growing in a laboratory growth medium. The typical practice is to prepare an enrichment culture, which is to prepare a culture growth medium that will favour the growth of an organism of interest. A sample such as food, water or a bodily fluid that may contain the organism of interest is introduced into the enrichment culture medium. Typically, the enrichment culture medium is an agar plate where the agar medium is enriched with certain nutrients. Appropriate conditions of temperature, pH and aeration are provided and the medium is then incubated. The culture medium is examined visually after a period of incubation to determine whether there has been any microbial growth. It could take several days to obtain results.

Paper test strips including test reagents such as antibodies, are also used to determine whether a particular pathogen is present in a sample. This type of test simply provides a positive or negative result. It does not provide information about the quantity of pathogen that may be present. Another drawback is that paper strip tests have low sensitivity. Therefore there is a risk that a pathogen may be present below a level sufficient for the test to detect its presence.

Contamination of water supplies also causes illness and death. The United States Environmental Protection Agency has determined that the level of E. coli in a water supply is a good indicator of health risk. Other common indicators are total coliforms, fecal coliforms, fecal streptococci and enterococci. Water samples are currently analyzed for these microorganisms with membrane filtration or multiple-tube fermentation techniques. Both types of tests are costly and time consuming and require significant handling. They are not, therefore, suitable for field-testing.

Many disease conditions, such as bacterial and viral infections, many cancers, heart attacks and strokes, for example, may be detected through the testing of blood and other body fluids, such as saliva, urine, semen and feces for markers that have been shown to be associated with particular conditions. Early and rapid diagnosis may be the key to successful treatment. Standard medical tests for quantifying markers, such as ELISA-type assays, are time consuming and require relatively large volumes of fluid. There is also a serious need for the accurate and rapid identification of microorganisms and markers of the health of a patient.

The accurate, rapid detection and measurement of microorganisms, such as bacteria, viruses, fungi or other infectious organisms and indicators in food and water, on surfaces where food is prepared, on other surfaces which should meet sanitary standards is, therefore, a pressing need in industrial, food, biological, medical, veterinary and environmental samples. The routine inspection of industrial products for microbiologic contamination, and the early detection of micro-organisms which are not pathogenic but have a role in the determination of a product's shelf life, will also lead to more rapid release of safe products.

In a typical test assay, a fluid sample is mixed with a reagent, such as an antibody, specific to a particular analyte (the substance being tested for), such as an antigen. The reaction of the analyte with the reagent may result in a color change that may be visually observed, or in chemiluminescent, bioluminescent or fluorescent species that may be observed with a microscope or detected by a photodetecting device, such as a spectrophotometer or photomultiplier tube. The reagent may also be a fluorescent or other such detectable-labeled reagent that binds to the analyte. Radiation that is scattered, reflected, transmitted or absorbed by the fluid sample may also be indicative of the identity and type of analyte in the fluid sample.

In a commonly used assay technique, two types of antibodies are used, both specific to the analyte. One type of antibody is immobilized on a solid support. The other type of antibody is labeled by conjugation with a detectable marker and mixed with the sample. A complex between the first antibody, the substance being tested for and the second antibody is formed, immobilizing the marker. The marker may be an enzyme, or a fluorescent or radioactive marker, which may then be detected. See, for example, U.S. Pat. No. 5,610,077.

There are presently many examples of one-step assays and assay devices for detecting analytes in fluids. One common type of assay is the chromatographic assay, wherein a fluid sample is exposed to a chromatographic strip containing reagents. A reaction between a particular analyte and the reagent causes a color change on the strip, indicating the presence of the analyte. In a pregnancy test device, for example, a urine sample is brought into contact with a test pad comprising a bibulous chromatographic strip containing reagents capable of reacting with and/or binding to human chorionic gonadotropin (“HCG”). The urine sample moves by capillary flow along the bibulous chromatography strip. The reaction typically generates a color change, which indicates that HCG is present. While the presence of a quantity of an analyte above a threshold may be determined, the actual amount or concentration of the analyte is unknown.

In order to quantitatively measure the concentration of an analyte in a sample and to compare test results, it is advantageous to use a consistent test volume of the fluid sample each time the assay is performed. The analyte measurement may then be assessed without having to adjust for varying volumes. U.S. Pat. No. 4,088,448, entitled “Apparatus for Sampling, Mixing the Sample with a Reagent and Making Particularly Optical Analysis”, discloses a cuvette with two planar surfaces defining a cavity of predetermined volume for receiving a sample fluid. The fluid is drawn into the cavity by capillary force, gravity or a vacuum. The sample mixes with a reagent in the cavity. The sample is then analyzed optically. There is no convenient location for placement of the sample on the disclosed device. The open side of the cavity is brought into contact with the sample, possibly by dipping the open side into the sample. There is also no separation medium incorporated in the device. If separation is required, it must take place prior to drawing the sample into the device.

In U.S. Pat. No. 4,978,503, entitled “Devices for Use in Chemical Procedures”, a device is shown including upper and lower transparent plates fixed together in parallel, opposed and spaced relation by adhesive to form a capillary cell cavity. The cavity is open at opposite ends. One open end is adjacent to a platform portion of the lower plate for receiving the sample. The other open end allows for the exit of air. Immobilized test reagents are provided within the cavity, on inner surfaces of one or both plates. The reaction between the sample and the reagent may be detected optically, from one of the open ends of the cavity. Filter paper may be provided on the platform to restrict the passage of red blood cells into the cavity, for testing blood. In one embodiment, plastic arms support the plates. Removable handles are also provided for use during various stages of the use of the device. The disclosed devices appear to be complex to manufacture and use.

U.S. Pat. No.6,197,494 B1, entitled “Apparatus for Performing Assays on Liquid Samples Accurately, Rapidly and Simply”, discloses assay devices comprising a base, an overlay defining a receiving opening, a reaction space and a conduit connecting the opening to the space, and a cover also defining a sample receiving opening and a viewing opening. When assembled, the sample receiving openings are aligned and the viewing opening is positioned over the reaction space. Heat sealing, solvent bonding or other appropriate techniques may be used to connect the layers to each other. Light may be provided through any of the layers, which act as waveguides, for optical analysis of the sample. By providing the light through the edge of the overlay, for example, light scattered, transmitted or absorbed by the sample may be detected by appropriate placement of standard detectors. By providing the light through the base or cover, fluorescence of the sample may be detected. Light may pass through the reaction space transverse to the layers, as well. Light passing through the reaction space may also be reflected off a layer, back through the reaction space. The disclosed devices comprise at least three pieces that require assembly. A simpler device would be desirable.

More recently, Chin and Wang have been granted a patent (U.S. Pat. No. 6,197,599) that describes a method for detecting proteins using protein arrays. This patent describes a method for qualitatively looking at protein-protein interactions between a cell lysate and a known set of proteins. However, this method however does not provide a quantitative method that will measure the concentration of specific analytes contained within various test samples.

There is therefore a need for a rapid and efficient methodology for determining the presence of analytes in a sample and for determining the quantity of respective analytes in the sample. There is a need for an assay device that permits a user to carry out the methodology in an efficient and user-friendly manner.

### SUMMARY OF THE INVENTION

The present invention includes a method of rapidly detecting the presence of analytes in a sample. Quantitative and qualitative measurements of analyte concentration in a sample may also be rapidly obtained.

According to a method of the present invention, the sample may be subjected to a force application means for the controlled progressive fragmentation of any matrix analyte, which is preferably a pathogen present in the sample, into a plurality of fragments. The sample is then introduced into a vessel that contains reagents that rapidly bind to the fragments of the analyte(s) to which the assay is directed. The sample is then introduced to an assay device that has a loading area, a separating area and a reading area. The sample is introduced into the loading area of the assay device and moves through the separating area to the reading area preferably by capillary action. The methodology permits for the detection of analyte fragments in less than thirty minutes.

According to another aspect of the present invention, a method of rapidly detecting the presence of an analyte in a sample is provided wherein a sample including the analyte and analyte metabolites produced by the matrix analyte are introduced into a vessel that contains a reagent or reagents that rapidly bind to the analyte and to the metabolite. The sample is then introduced to an assay device that has a loading area, a separation and a reading area. The sample is introduced into the loading area of the assay device and moves to the reading area preferably by capillary action. The methodology permits for the detection of analytes and metabolites.

The invention further includes an assay device for determining the presence of an analyte in a sample. The assay device may include a means for transferring the sample and/or a filter for separating unwanted components from the sample greater than a predetermined size in a fluid component of the sample.

According to one aspect of the present invention, the device has loading, separation and reading areas. The assay device defines a chamber between the loading portion and the reading portion such that a liquid portion of the sample moves from the loading portion to the reading portion by capillary action. At least one test dot is printed on the reading portion. The test dot includes a bound reagent that is adapted to bind to analyte fragments of the analyte for which the assay is directed. Once the fragments are bound to the test dot, the presence of the analyte fragments in the test dot can be determined by methods known in the art. The test dot may alternatively include a bound reagent that is adapted to bind to analyte or other metabolites that are produced by an analyte which is a bacterium or other pathogen to which the test is directed. The reading portion may also have a section for gathering analyte labeled with detectable markers for visual detection.

According to another aspect of the invention there is provided a device for assaying a sample for the presence of an analyte, the device comprising:
A loading portion for receiving a quantity of the sample;
a chamber, said chamber being defined by two non-contiguous surfaces;
said chamber having a first end in fluid communication with the loading portion and a second end spaced from the first end, said non-contiguous surfaces being separated by a distance sufficient to create capillary flow of said sample into said from said loading portion;
a reading portion in fluid communication with said second end of the chamber, the reading portion having printed thereon a test dot for detecting the presence of an analyte, the test dot including a reagent for binding the analyte.

According to yet another aspect of the present invention there is provided a method of detecting the presence and quantity of an analyte in a sample comprising the following steps:
Obtaining the sample;
Combining the sample with a solution to produce a sample solution;
applying a force application means to the sample solution for exploding the analyte into a plurality of analyte fragments;
labelling the analyte fragments with a detectable marker;
applying a measured volume of the sample solution to an assay device that is adapted to display an indication of the presence of said analyte fragments; and
detecting a signal intensity of the labelled analyte fragments with a detecting means.

According to yet another aspect of the present invention there is provided a method of matrix format comprising the following steps:
Obtaining the sample; and
applying the sample to an assay device that is adapted to display an indication of the presence of said analyte(s); and
reading the analyte(s) as a random array format; and
printing and reading the analyte(s) to be measured in a fixed array format; and
printing and reading the analytes in a hybrid format, consisting of both fixed arrays as well as random arrays.

According to yet another aspect of the present invention there is provided a method of detecting the presence and quantity of an analyte in a sample comprising the following steps:
Obtaining the sample;
Incubating the sample for a period of time;
Combining the sample with a solution to produce a sample solution;
labeling the analyte with a detectable marker;
applying a measured volume of the sample solution to an assay device that is adapted to display said labeled analyte; and
detecting a number of labeled analyte units with a detecting means.

### BRIEF DESCRIPTION OF THE DRAWINGS

In drawings which illustrate by way of example only a preferred embodiment of the invention,

FIG. 1 is a top view of an assay device of the present invention;

FIG. 2 is a top view of an assay device of the present invention for carrying out a fixed array test;

FIG. 3 is a microscope photograph of a top of an assay device of the present invention for carrying out a fixed array test;

FIG. 4 is a graph showing a relationship between fluorescent intensity of test dots and known antigen concentration in a sample;

FIG. 5 is a graph showing a relationship between fluorescent intensity of calibration dots and the amount of antigen in the calibration dots;

FIG. 6 is a graph showing a relationship between the antigen concentration in the sample and the amount of antigen in the calibration dots;

FIG. 7 is a graph showing a relationship between the log of the fluorescent light reading and concentration of analyte;

FIG. 8 is a microscopic image of yeast particles labeled with fluorescent enzymes;

FIG. 9 is a graph showing the fluorescent intensity of various samples comprising a florescent dye conjugated to a specific metabolite of a micro-organism;

FIG. 10 is a microscopic image of fluorescently labeled bacteria;

FIG. 11 is a microscopic image of two pre-printed capture spots of the present invention with attached and pre-printed bacterial fragments;

FIG. 12 is a microscopic image showing the dynamic concentration and capture of fluorescent E. coli bacteria on the surface of two preprinted capture dots;

FIG. 13 is a graph showing a correlation between expected and calculated antigen concentration in a sample of the antigen; and

FIG. 14 is a microscopic image showing an assay device of the present invention having vertical arrays of calibration dots and test dots printed thereon.

### DETAILED DESCRIPTION OF THE INVENTION

The present invention relates to a method of rapidly determining the presence of analytes in a sample and a device for carrying out the method. The analyte detected according to the present invention can be a pathogen. The present invention reliably detects pathogen contamination in a sample within thirty minutes. This advancement significantly benefits the food industry where perishable items need to be tested and delivered to stores and restaurants as soon as possible. The invention can be directed to different types of samples that can be infected by a pathogen including water supplies, human blood, cells, tissues, fluids and secretions.

Three preferred embodiments of the present invention are described herein. These are 1) Random array; 2) Fixed array; and 3) Hybrid array.

### Random Array

According to the random array method, a sample is obtained for analysis as to whether the sample has been contaminated with a pathogen. For example, the pathogen can be a strain of bacteria that, following ingestion, is pathogenic to humans. Examples of such bacteria are E. coli O157:H7, Salmonella, Listeria species, Listeria monocytogenes and Campylobacter jejuni. The method also detects other microorganisms, including viruses, yeast, mould and other infectious organisms.

The sample is incubated using industry accepted enrichment media such as CASO broth to grow enough pathogen organisms to ensure that there is a minimum of log4 pathogen colony forming units (CFU) per ml of sample fluid. The enrichment period is normally at least 18 hours. This time can be reduced to hours by providing an enrichment medium. Several enrichment media known in the art can be employed.

According to the random array method, a calibrated amount of the enriched sample is drawn before analysis, into an adjunct vessel containing labeling reagents. The adjunct vessel is preferably a syringe type applicator. An additional amount of air is also drawn into the adjunct vessel. The vessel contains reagents for binding to the analyte to be assayed. Preferably, the reagents are lyophilized antibodies that reconstitute immediately and instantaneously upon contact with the liquid sample. The instantaneous reconstitution of the preferred lyophilized antibodies also avoids clumping or lumping of the sample. Other reagents known in the art may also be used.

The reagents may be labeled with a fluorescent, chemical, calorimetric, heavy metal, radioactive, enzyme specific label, or other detectable labels known in the art. Preferably, a pathogen specific antibody is labeled with a fluorescent dye marker in the adjunct vessel. The dye preferably has a specific wavelength. The adjunct vessel preferably also has an additional dye that provides the operator with visual confirmation that the sample reading area of the assay device is correctly flooded with test sample. The preferred dye is bromophenol.

The adjunct vessel may also contain a concentrating material for concentrating liquid from the sample thereby concentrating the analyte in the sample. The concentrating material may be any material that absorbs fluid and does not react with the analyte in the fluid sample. Superabsorbant polymers, such as polyacrylates, cellulose derivatives and hydrogels, for example, are preferred. A suitable commercially available superabsorbant polymer is Favor®-Pac 100 (Stockhausen Inc., Greensborough, N.C., USA), a cross-linked polyacrylic acid and grafted copolymer. The carboxylic groups of the polymer are solvated when brought into contact with water and absorb aqueous fluid. Thirty milligrams of Favor®-Pac 100 in 300 to 350 microliters of fluid, was found to increase analyte concentration by a factor of three.

The sample is preferably incubated for about five minutes in the adjunct vessel. During this time the fluorescent dye labeled antibodies bind to the pathogen organism that is the analyte. Once the incubation period is completed, the operator preferably discards the first two drops from the adjunct vessel.

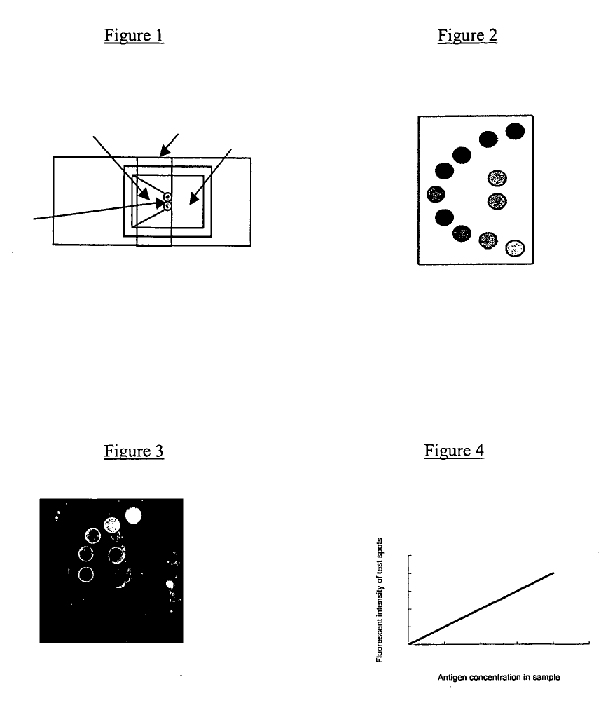

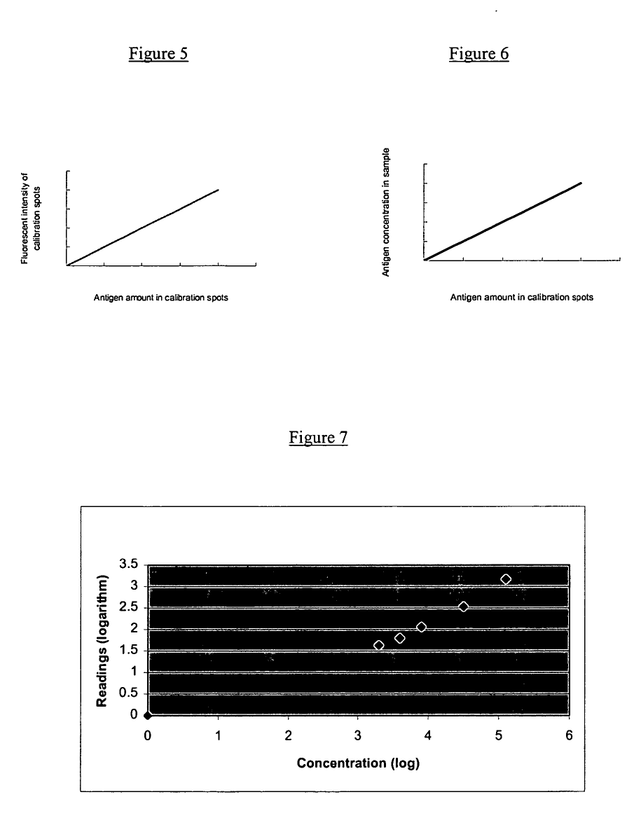

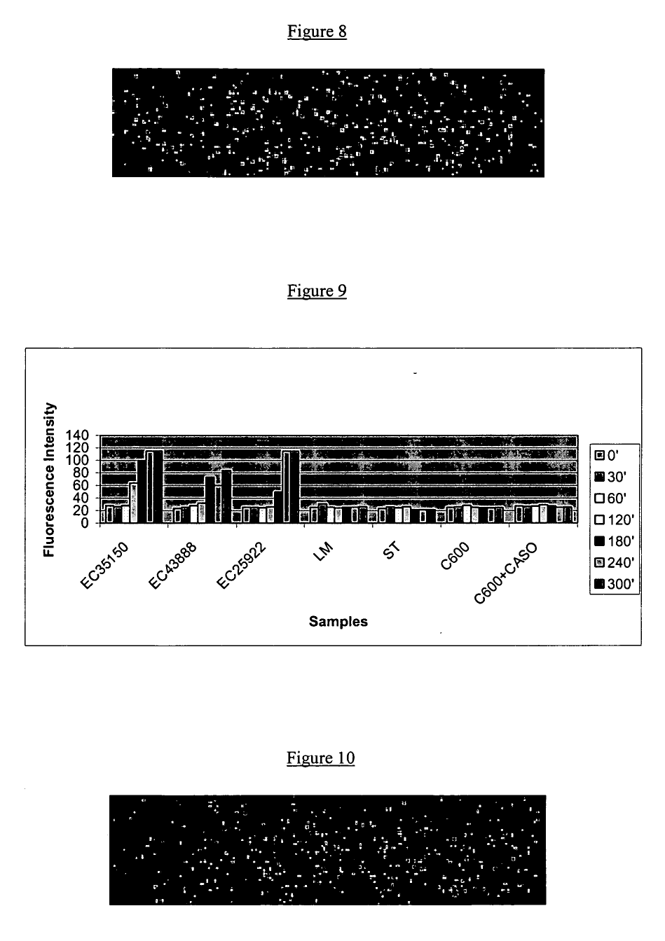

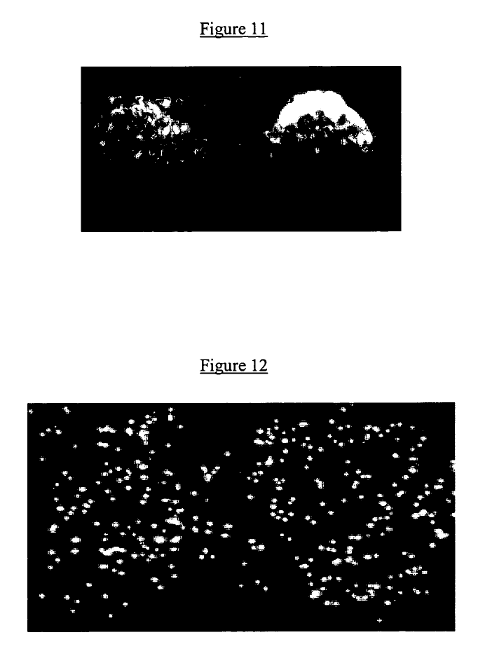

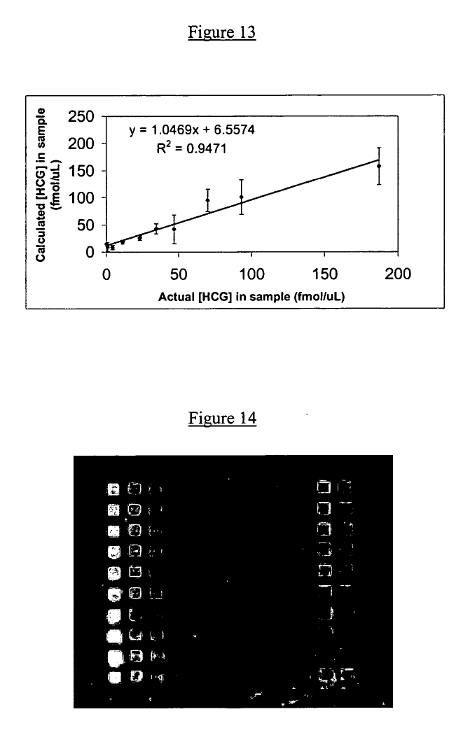

A third drop of the sample fluid is then applied to an assay device. A preferred assay device 10 for carrying out the random array method is described in FIG. 1. The assay device 10 has a sample loading area 12, a separation area 14, a lid 18 that covers the sample loading area 12, a separation area 14, and a sample reading area 16. A preferred separating area is a medium that is a collection of microspheres or beads which, when exposed to fluid, move and transiently abut each other. The interstitial spaces or pores between the microspheres are also, therefore, transient. It is believed that the fluid is drawn through the interstitial spaces between the microspheres by capillary force. Such a separating medium is therefore referred to as dynamic capillary filter.

Providing the separation medium within the assay device 10 simplifies the testing process by eliminating the need for a separate separation step prior to application of the sample to the assay device 10. This enables the assay device 10 to be used at the point of patient care, for example, by the patient, at the patient's bedside or in a doctor's office. In food and environmental testing, the assay device can also be used in the field, at the source of the sample. In addition, the microspheres of the present invention provide improved fluid flow without restriction by the fiber in the chromatographic paper or other fibrous materials used in the prior art to wick the fluid component of a biologic sample away from the cellular component.

While incorporating the separation medium in the assay device 10 is one advantage of the present invention, there may be times when a separate filtration step is preferred. Separation may be provided by centrifugation, for example. It may also be advantageous to concentrate the analyte by centrifugation. Centrifugation has been used for the concentration of bacteria, for example. Iumunomagnetic bead concentration and separation techniques can be used to concentrate bacteria and to separate the bacteria from unwanted components of the fluid sample. Certain water samples may not need filtration, either. Whether filtration is required or not, providing the microspheres in the separation area 14 is still preferred, because it has been found that the microspheres improve the fluid flow through the assay device 10.

A plurality of positive control dots is preferably printed on an underside of the assay device 10. There are preferably 6 positive control dots. The positive control dots are printed on to the assay device with the analyte of interest—typically a bacterial pathogen—bound to the surface of the assay device in the positive control dots. During the fluid transfer phase, loose analyte-specific antibody—fluorescent dye conjugates will bind to the captive analyte in the positive control dots to provide a positive control for the analyte detection test.

To use the assay device 10 in accordance with the present invention for the random array test, preferably the third drop of the sample fluid from the adjunct vessel is placed in the loading area 12. The fluid sample may be about 5 micro liters to about 65 micro liters, for example, depending on the size of the separation area 14. Preferably, the amount of the fluid sample applied is greater than the volume of the separation area 14 by a sufficient amount so that after filtration, there is still excess fluid sample in the loading area. This helps slow the evaporation of the fluid sample from the loading area 12. The lid 18 is then preferably slid over the loading area 12 and the separation area 14 and secured in place, exposing the reading area 16 and securely covering the loading area 12 and the separation area 14. The fluid sample is drawn through the separation area 14 and through the microspheres, if present, by capillary force and gravity to remove materials over a predetermined size. The filtered fluid sample exits the separation area 14 at the entrance of reading area 16.

In other implementations of the invention, the fluid sample may be drawn directly from a source, such as from a water supply or a bodily fluid and may be applied by known techniques, such as a pipette to the loading area of the assay device. A syringe may also be used. A drop of blood could be applied directly from a pinprick to the loading area 12. The fluid sample may also be drawn from a culture medium.

The reading area 16 is preferably colorless or transparent. Once the sample fluid reaches the reading area 16, the sample fluid in the reading area will include the following: 1) pathogen organisms conjugated with a fluorescent dye; 2) sample fluid preferably dyed blue for confirmation that the sample viewing area was correctly filled; and 3) loose pathogen-specific antibodies conjugated with fluorescent dye. The loose pathogen-specific antibodies conjugated with fluorescent dye will bind to the test dots to indicate a positive test.

Fluorescent, chemiluminescent, bioluminescent calorimetric, or other reaction products that indicate the presence of the analyte can be detected by techniques well known in the art. For example, the labeled pathogen organisms may be read visually, under a microscope. A photoconductive detection device, such as a photodiode, a photomultiplier or a CCD, may also be used. A detecting device, such as a spectrophotometer, a luminometer, a fluorometer or another appropriate detector coupled to a reader may also be used, as is known in the art. The intensity of the reaction product may be measured to determine the amount of analyte present in the sample by comparison to calibration curves.

The assay device 10 may be designed to be read by a portable spectrophotometer which reads, for example, the change in color after the analyte has reacted with the labeled antibody. A Genepix Spectrophotometer, available from Axon Instruments, Inc., Foster City, Calif., U.S.A., may be used, for example. Once the spectrometer, or other such detector, has performed the necessary data calculations, the results are transmissible by digital transmission over the telephone lines, by cell phone, or other computer network system.

The detector may be moved with respect to the reading portion 16 or the reading portion 16 may be moved with respect to the detector, automatically or manually.

Fluorescent emissions from a fluorescently labeled analyte may be detected using a fluorometer. Information about the distribution of fluorescent emissions, including location and intensity, can be obtained by acquiring an image using a CCD camera and commercially available software, such as microassay analysis software, such as GenePix Pro.TM. from Axon Instruments, Inc. Image-Pro.TM. 4.1, available from Media Cybernetics, Silver Spring, Md., U.S.A., is useful for counting fluorescently labeled bacteria.

In another alternative, changes occurring during an antibody/analyte reaction may be detected or measured by changes in radio frequency if a radio frequency sensor (not shown) is incorporated into one of the plates of the assay device 10.

The assay device 10 is preferably discarded after use, following appropriate, standard hazardous waste guidelines.

In counting the number of organisms contained in an aliquot of sample solution, only labeled organisms are counted. The concentration is expressed as the number of organisms contained in a known fluid volume.

### Fixed Array and Hybrid Array

The fixed array method detects the presence and concentration of specific proteins including bacterial or other microbe fragments and bacterial or other microbe metabolites.

The fixed array method includes the step of breaking up the analyte, which is typically bacterial cells or other pathogens in the sample, into a plurality of pieces or fragments. The breaking up of cells is accomplished through a process of controlled progressive fragmentation of the cell membrane. The cell membrane is broken into fragments and the membrane is resultantly separated from the contents of the cell.

A force application means is used to apply the required force to accomplish the controlled progressive fragmentation. This is a time and energy dependent procedure, including microwave irradiation. The force application means is preferably a transfer of ultrasound energy. A sonic probe is preferably inserted into a vessel containing the sample and oscillated at a predetermined tuned frequency dissipating 20 kHz at a variable power dissipation of 50 to 475 Watts with the preferred application time range of 60 to 250 seconds. The sonic probe may be but is not limited to the 550 Sonic Dismembrator, of Fisher Scientific. Other force application means known in the art for fragmenting bacterial cells such as microwaves, enzymes such as proteolytic enzymes, electrical energy, and laser heat dissipation may also be employed for the purposes of the present invention. This step essentially multiplies the amount of antigen label binding sites that can be tested in the sample without incurring the delay that results from waiting for bacterial or other pathogen cells to multiply.

A dismembrator is used in a preferred protocol for breaking bacteria into fragments to be stained with a label-conjugated antibody following sonication. This protocol provides increased sensitivity and shorter time for a bacterial test. Sonication buffer and CASO broth are used to dilute bacteria which may be E. coli O157 # 35150, and anti-α O157 antibody conjugated to Alexa Fluor® 594 (Molecular Probes, USA) is used for staining bacteria and bacterial fragments. Bacterial culture is diluted to 100,000; 10,000; and 1000 bacteria per 1 ml. 1 ml of each sample is sonicated in a siliconized tube. Anti-α O157 antibody (1:100) is used for staining. Samples are observed under fluorescent microscopy. Fragments are effectively obtained by using 425 Watts of ultrasonic vibration energy from 30 to 90 seconds.

According to the fixed array method, a calibrated amount of the sample is drawn into an adjunct vessel before device analysis. The adjunct vessel contains the labeling reagents as described above for the random array method. The adjunct vessel therefore preferably includes protein specific antibodies conjugated with a specific wavelength dye and an additional dye that provides the operator later with visual confirmation that the assay is proceeding. The adjunct vessel is preferably a syringe type applicator

The sample is preferably shaken in the adjunct vessel for ten seconds and then preferably incubated in the adjunct vessel for about five minutes. The protein analytes of interest are tagged with the conjugated antibodies during the incubation period. Once the sample has been exposed to the reagent for a sufficient amount of time, the reacted sample is then delivered from the adjunct vessel to an assay device of the present invention.

The fixed array assay device employs the same assay device as shown in FIG. 1. As shown in FIG. 2, the reading portion of the fixed array assay device has printed thereon at least one and preferably at least two test dots 20. More preferably, a plurality of dots for detecting the presence of the analyte are printed on the reading area 16. The test dots include a reagent that specifically bind to the analyte. The reagent is preferably a bound antibody specific for the analyte. The bound antibodies are preferably spaced apart to make each bound antibody available for binding to the test antigen free of stearic hindrance from adjacent antigen complexes. Preferably, a non-reactive protein separates the bound antibodies in the test dots.

The reading area 16 preferably has calibration dots 22 printed thereon. The calibration dots include a pre-determined amount of said analyte for reacting with unreacted reagent form the vessel that is bound to a detectable marker. The calibration dots allow the intensity of the label to be correlated to the amount of the antigen present. The intensity of label in the test dots can then be used to derive the quantity of antigen present.

The test dots are suitable for detecting the presence of very small protein fragments in the range of for example, 7-10 nanometers. These small fragments correspond to bacterial cell membrane fragments that result from the controlled progressive fragmentation process. The test dots are also appropriate for binding to proteins and other by-product metabolites that are produced by bacteria in a sample. However, the bacteria, which are typically 1-7 μm in length or width, are also able to concentrate by binding to the bound antibodies in the test dots.

The reading area 16 may optionally also have a zone for receiving an amount of analyte bound to labeled antibody that has not bound to a test dot. This labeled analyte can be detected by microscopic means or other detection means. Calculations as to the quantity of pathogen present can also be made for a given volume of sample detected. The number of particles bound to a detectable label can be counted. The volume of sample can be pre-determined so that a calculation of number of particles per unit volume can be carried out. This assay device is a hybrid array assay device. The device allows a user to calculate the amount of analyte present using both the fixed array dots and the random array methodology of counting the amount analyte present per unit volume of sample fluid by counting the number of labeled particles by visual means.

The hybrid array assay device has test dots printed thereon that preferably contain bound antibodies that are specific for a particular bacterial protein or metabolite produced by a bacterial pathogen of interest. This assay device also has a reading portion for gathering bacteria labeled with a detectable marker. The assay device is thus configured to display both the presence of antigen proteins and metabolites produced by microorganisms of interest and the presence of the intact microorganisms. This methodology is referred to as hybrid array. This provides a sensitive and reliable test. According to this hybrid array method, it is not strictly necessary to fragment the bacteria. The sample potentially including bacteria is preferably exposed to an enriched growth medium. The sample is then introduced to the adjunct vessel having antibodies to the antigens of interest bound to a detectable marker. The sample is then delivered from the vessel to the assay device.

Where the fixed array or hybrid array tests are directed to cells, micro-organisms proteins and metabolites, the test is not limited to testing for the presence of one protein but may be specific for a broad array of antigens, proteins and metabolites. Hence, the fixed array assay device and the hybrid array device may have additional collections of test dots and calibration dots for several different analytes printed thereon. This allows tests for several different types of pathogens or other analytes to be carried out simultaneously.

The device also allows for the display and reading of tissue micro-arrays. The micro-arrays, which are made by depositing and attaching tissue sections directly onto the base component of the device, can be unstained, pre-stained or stained while in the device. Secondary labeling for the detection of antigens, known in the art, is then accomplished either in the device, or before the tissues are attached to the base. Labeling methods include use of immuno staining, particles, enzymes, dyes, stains, and other fluorescence and density markers.

After incubation in the adjunct vessel, the test operator preferably discards the first two drops from the adjunct vessel. The operator then dispenses a third drop into the loading area 12 of the assay device 10. The sample fluid is drawn through the separation area where sample impurities are preferably filtered out. The sample fluid then passes into the reading area. At this stage, the sample fluid in the reading area will include 1) proteins conjugated with a fluorescent dye; 2) sample fluid preferably dyed blue for confirmation that the sample viewing area was correctly filled; and 3) loose protein-specific antibodies conjugated with fluorescent dye.

The laminar flow of the sample fluid then causes the test fluid to be drawn past and exposed to the calibration dots containing varied concentrations of the protein analyte of interest and the test dots containing capture antibody. The principal of operation is that the loose fluorescing antibodies are attracted to the calibration dots and provide a basis for automatic calibration of the test. The protein-fluorescent dye conjugates are captured by the test dots.

The fixed array assay device and the hybrid array assay device are both preferably read by a microscope that is operated by a computer. The microscope takes readings of light intensity that are processed by a computer which calculates the amount of an analyte present based on these readings.

Other means known in the art including those discussed above for the random array device may be employed for determining the amount of analyte present in the test dots. The calculation of the quantity of analyte present may be accomplished by way of calibration curves.

To determine the concentration of analyte in a sample, the concentrations of two characteristic assay reagents are predetermined. A relationship between a fluorescent intensity of the fixed test dots in a series of samples with known antigen concentrations is determined. An example of a relationship between fluorescent intensity of test dots and known antigen concentration is a sample is shown in the form a a graph in FIG. 4. Next, a relationship between fluorescent intensity of the calibration dots and the amount of antigen in the calibration dots, determined by using excess detection antibody, is shown in FIG. 5. From FIG. 4 and FIG. 5, an association between the antigen in the sample and the antigen dot concentration is determined as shown in FIG. 6. This calibration curve serves as a batch-specific standard curve for the determination of the antigen concentration in the samples.

In the instance of a sample of unknown antigen concentration, the sample is premixed with an excess of detecting antibody. This solution is applied to an assay device such as the assay device shown in FIG. 2. The fluorescent intensity of the test dots is normalized against the calibration curve for that particular analyte to provide a normalized test dot value. This normalized test dot value is then read off the calibration curve shown in FIG. 6 for that analyte to give the concentration of analyte in the sample.

The reading area of the device may also be loaded with portions of chromatography substrate, such as paper or gels. The separation of proteins may be advantageously displayed and labeled to be read. Respective concentrations of proteins is then measured by fluorescence quantitation when compared to a calibration sample.

The assay device is preferably discarded after use.

### EXAMPLES

### Example 1

### Quantitative Detection of Bacteria using Random Arrays

i. Bacteria—Random Array.

Escherichia coli O157, including O157:H7 and other O157 enterohaemorragic Escherichia coli (EHEC) strains are found in solid or liquid food samples. The random array assay device provides a rapid, convenient and sensitive method based on immunofluorescent staining, separation and detection technology that isolates bacteria from food particles, to be counted in the reading area of the device. Results are determined by counting the number of antibody labeled and stained bacteria, randomly arrayed, using a microscope operatively connected to a computer for processing images hereinafter referred to as “the reader”.

Each device preferably includes a control dot in the reading area that preferably containing goat anti-mouse IgG. This will bind the mouse anti-E. coli O157 antibody conjugated with fluorescent dye contained in the vessel used to load the sample into the loading area of the device. Regardless of whether any E. coli O157 is present in the sample or not, this dot is always detected as a fluorescent emission, thus ensuring that all facets of the test have been successfully completed.

When testing samples, the performance of the reagents and methodology is periodically evaluated by testing positive and negative controls.

ii. Bacteria—Random Array Detection Matrix

Current culture pathogenic E. coli O157:H7 ATCC#35150 in 1% bovine serum albumin serial dilution, were made at log7, log6 and log3 concentrations. Random detection matrices were prepared using capture antibody at 0.12 mg/ml in 0.05 molar sodium carbonate/sodium bicarbonate, pH 10.5. The devices were blocked with 1% bovine serum albumin. The entire reading area of the random array assay device was coated with the detection matrix. The test log concentrations, labeled with fluorescent antibody, (as in i. above), were introduced into the device via the loading area and the samples read and counted. Control dilutions were plated for accuracy comparison. FIG. 7 shows the corresponding plot.

iii. Mold and Yeast—Random Array.

The specific quantitative detection of mold and yeast is carried out according to the present invention. The yeast particles are first processed in the vessel, which contains a fluorescent enzyme specific for binding only to the chitin expressed in the surface coat of the yeast spores. The labeled spores are loaded into the device, as previously described. An example of the reading area that displays individual labeled yeast spores is shown in FIG. 8.

The bright particles, as displayed in FIG. 8, are counted in the reader. As the volume of carrier fluid in the reading area is accurately predetermined, the ratio of number of spores per volume reflects the actual concentration of spores in the test sample. Mold is enumerated in the device, using similar methods.

iv. Metabolite Concentration—Background Fluorescence Intensity.

A further example is illustrated in FIG. 9, based on using a fluorescent dye conjugated to a specific metabolite produced by the micro-organism to be detected, in this case coliform bacteria. In FIG. 9, EC represents coliform species, LM, ST represents non-coliforms and C represents metabolite only.

The actual concentration of metabolite is measured by the intensity of the background fluorescence measured in the reading area of the device. The measured intensity is compared to a known, pre-test calibration curve, which is converted to the respective concentration of coliforms in a known volume of test sample.

v. Total Viable Count (TVC) Bacteria.

For testing and quantitation of the total number of viable bacteria in a test sample, Campylobacter were grown in YM broth. A test sample was aspirated into a reaction vessel and allowed to react with fluorescence specific nuclear dye (Syto 61, Molecular Probes, Eugene, Oreg., USA). Following 5 minutes of staining time, the sample was loaded into the device and the concentration of bacteria determined in the reading area of the device as shown in FIG. 10. FIG. 10 illustrates the number of bacteria in the test sample to have a concentration of 6.3 log6.

vi. TVC with Random Matrix Concentration.

Random Matrix concentration is shown as an example demonstrating that concentration by selective filtration may be used to substantially increase a very low number per volume of cells to a much higher number of cells, thereby significantly decreasing time for detection and counting of cells. Table 1 clearly demonstrates the advantage of combining a concentration means with the device.

TABLE 1
Bacteria Concentration for TVC Readings

| | | | | Concen- | | Equivalent |
|---|---|---|---|---|---|---|
| | | | Run | tration | TVC | Concen- |
| | | Filtered | Time | To Detect | Reading | tration |
| Filter | Fluid | Volume | Minutes | per ml | Device | per ml |
| Single | Tap | 3000 ml + | 90 | 10¹ | 72 | 2.1 × 10⁴ |
| Unit | Water | spike | minutes | | | |
| | | of | | | | |
| | | 30,000 | | | | |
| | | E. coli | | | | |

Cell concentrations as low as 1 bacterium per milliliter are detectable in the reading area of the device.

### Example 2

### Quantitative Detection of Organisms by Fixed Immuno Matrix Assay

i. Bacteria—Fragments.

Random array assays allow accurate determination of whole or large particle count. Fixed array assays on the other hand allow for the capture or increase in surface area density of proteins, aggregates of proteins, membrane fragments of organisms on matrix capture dots pre-printed on the reading area of the assay device.

The advantage conveyed by using this aspect of the method lies in the ability to detect lower concentrations of specific fragments as a function of fluorescence intensity.

FIG. 11 shows two preprinted capture dots with attached and concentrated bacterial fragments, which would otherwise not have been detected.

ii. Bacteria—Whole Bacteria Assay.

Another aspect of the method is demonstrated in the FIG. 12.

Preprinted capture antibody matrix dots are also used to capture whole fluorescent cells as they bind with the respective capture antibody. This assay has the added advantage in that dynamic flow particle capture and enumeration may be carried out. FIG. 12 shows the dynamic concentration and capture of fluorescent E. coli bacteria on the surface of two preprinted capture dots. Each individual bright dot results from a single bacterium. The faint, circular background defines the two capture dots.

### Example 3

### Quantitative Detection of Soluble Proteins by Fixed Immuno Matrix Assay

This example describes the immuno matrix assay method for the quantitative analysis of an antigen. Two sets of protein arrays are printed on the surface of the device: calibration dots, with varied concentrations of the antigen of interest, and test dots, which contain the capture antibody. The sample and an excess of detecting antibody are loaded into the device. The fluid fills the reaction chamber by capillary action. The amount of antigen in the sample is quantified by normalizing the fluorescence intensity of test dots to the calibration dots. This value is then converted to the amount of antigen in the sample using a predetermined, batch-specific equation.

In contrast, conventional immunoassays, such as RIA and ELISA, are usually time-consuming and demand expert skills from the operators. Furthermore, conventional immunoassays require relatively large volumes of sample for analysis (100-1000 μL). Using the immuno-matrices and quantification method, a fully quantitative analysis can be provided within minutes using a single device and less than 20 μL of sample, which provides a significant advantage over any existing system.

The method and device were tested for the immuno-matrices quantitation of hCGβ (human chorionic gonadotrophin-β). hCGβ was used for the calibration dots, monoclonal anti-hCGβ antibody M94139.7 was used as the capture antibody and AlexaFluor 660-labeled anti-hCGβ antibody M94138 as the detecting antibody (Fitzgerald Industries, Mass.). The mean of six experiments is presented in FIG. 13. This data shows an excellent correlation between the expected and calculated antigen concentration in the sample, with a line equation of y=1.0469x+6.5574 and R=0.9732 (For a perfect test, the line will be y=x).

### Example 4

### Quantitative Detection of Multiple Soluble Proteins by Fixed Immuno Matrix Assay

The method and device also is used for the detection and quantitation of soluble proteins in a variety of fluids, including antigens found in point-of-care tests including medical, veterinary and environmental applications. The added advantage is that each device has a calibration matrix printed in the reading area. FIG. 14 illustrates a Fixed Immuno Matrix supported by the calibration matrix.

The 2 vertical arrays on the right to left of FIG. 14, are test dots which have captured similar concentrations of antigen from the test sample. The 6 vertical arrays, from left to right, have each array at decreasing known calibration concentrations. Each vertical array consists of 10 dots with similar amount of antibody label captured by the known antigen concentration. Each horizontal array with decreasing intensity constitutes the calibration matrix. The unknown test dots (2 arrays, right to left of FIG. 14) are then compared to the calibrated value in order to determine concentration of the unknown antigen.

This example confirms the reproducibility for measuring Human Chorionic Gonadotrophin protein concentration in the Fixed Immuno Matrix pre-printed in the reading area of the assay device, in the femto-gram per micro liter range (fmol/μL).

The assay device also contains the option for combining random arrays with fixed arrays displayed and read in the reading area of the device. This is referred to as hybrid array as discussed above.

Those skilled in the art will recognize, or be able to ascertain using no more than routine experimentation, many equivalents to the embodiments of the invention described specifically above. Such equivalents are intended to be encompassed in the scope of the following claims.

1. A device for assaying a sample for the presence of an analyte, the device comprising:
A loading portion for receiving a quantity of the sample;
a chamber, said chamber being defined by two non-contiguous surfaces;
said chamber having a first end in fluid communication with the loading portion and a second end spaced from the first end, said non-contiguous surfaces being separated by a distance sufficient to create capillary flow of said sample into said from said loading portion;
a reading portion in fluid communication with said second end of the chamber, the reading portion having printed thereon a test dots for detecting the presence of an analyte, the test dot including a reagent for binding the analyte.

2. A device according to claim 1 wherein the test dot binds antigens that are from about 7 nanometers to about 10 nanometers in length or width.

3. A device according to claim 2 further comprising a dynamic capillary filter located in the chamber, said dynamic capillary filter being in fluid communication with said loading and reading portions, the dynamic capillary filter including a plurality of particles, said particles being in a transiently abutting relation with one another and forming interstitial spaces therebetween;
whereby when said a fluid portion of said sample contacts said dynamic capillary filter, said fluid portion flows into said dynamic capillary filter, whereupon a fluid component of said fluid sample is separated from a non-fluid component of said fluid sample by passage through said interstitial spaces of said dynamic capillary filter and said fluid component thereafter flows over said reading portion.

4. A device according to claim 1 wherein the reagent is an antibody to the analyte.

5. A according to claim 4 wherein the analyte is a pathogen fragment, the antibody being labeled with a detectable marker.

6. An assay device according to claim 3 wherein the detectable marker is a fluorescent dye.

7. An assay device according to claim 1 further comprising a plurality of test dots being distributed on said reading portion.

8. An assay device according to claim 7 wherein the test dot includes bound antibodies that are separated by a non-reactive protein.

9. An assay device according to claim 8 wherein the bound antibodies bind antigens that are from about 7 nanometers to about 10 nanometers in length or width.

10. An assay device according to claim 1 further including at least two calibration dots printed on said reading portion, the calibration dots including a pre-determined amount of said analyte for reacting with said reagent.

11. An assay device according to claim 10 wherein the reading portion includes a positive control dot printed thereon for binding loose analyte specific antibodies.

12. An assay device according to claim 11 wherein the device includes a security dot printed thereon for verifying that the device is specific for a pre-determined type of assay.

13. An assay device comprising according to claim 1 wherein the reading portion further includes a sample reading area for collecting labelled unbound analyte.

14. An assay device according to claim 11 wherein the analyte is conjugated to a detection label,

15. An assay device according to claim 11 wherein the reagent is an antibody to the analyte.

16. An assay according to claim 12 wherein the analyte is a one of a pathogen fragment and a metabolite produced by the pathogen, the antibody being labeled with a detectable marker.

17. An assay device according to claim 16 wherein the detectable marker is a fluorescent dye.

18. An assay device according to claim 1 wherein the analyte is a pathogen.

19. An assay device according to claim 18 wherein the pathogen is selected from the group consisting of bacteria, viruses and fungi.

20. A method of detecting the presence and quantity of an analyte in a sample comprising the following steps:
Obtaining the sample;
Combining the sample with a solution to produce a sample solution;
applying a force application means to the sample solution for exploding the analyte into a plurality of analyte fragments;
labelling the analyte fragments with a detectable marker;
applying a measured volume of the sample solution to an assay device that is adapted to display an indication of the presence of said analyte fragments; and
detecting a signal intensity of the labelled analyte fragments with a detecting means.

21. A method according to claim 20 further comprising the step of calculating a quantity of analyte present in the sample based on said signal intensity.

22. A method according to claim 21 wherein the step of calculating a quantity of analyte present in the sample includes the following sub-steps:
detecting a signal intensity of a known concentration of labelled calibration-analyte in a solution with said detecting means;
calculating a ratio of the signal intensity of a concentration of labelled analyte fragments to the signal intensity of a known concentration of labelled calibration-analyte; and
calculating a concentration of the analyte present in the sample sample solution based on said ratio.

23. A method according to claim 20 wherein the detecting means is selected from the group consisting of a microscope, a photodiode, a photomultiplier, a CCD, a spectrophotometer, a luminometer, and fluorometer.

24. A method according to claim 20 wherein the force applied is selected from the group consisting of sonification, enzyme lysis, electrical energy, microwave and laser heat dispersion.

25. A method according to claim 20 wherein the step of labelling the analyte fragments with a detectable marker includes the following sub-step of combining the sample solution with a reagent that is adapted to bind to the analyte fragments to form a plurality of reagent-analyte fragment conjugates.

26. A method according to claim 25 wherein the step of combining the sample solution with the reagent is carried out in a vessel containing the reagent.

27. A method according to claim 26 wherein the vessel further contains a concentrating material.

28. A method according to claim 26 wherein the vessel is a syringe applicator.

29. A method according to claim 20 wherein the reagent is antibodies that bind specifically to the analyte.

30. A method according to claim 29 wherein the antibodies are lyophilized antibodies that are adapted to re-hydrate instantaneously upon contact with a fluid.

31. A method according to claim 20 wherein the detectable marker is a fluorescent dye.

32. A method according to claim 20 wherein the analyte fragments are from about 7 nanometers to about 10 nanometers in length or width.

33. A method according to claim 20 wherein the analyte is a pathogen.

34. A method according to claim 20 wherein the pathogen is selected from the group consisting of bacteria, viruses and fungi.

35. A method according to claim 35 wherein the analyte is a bacterium, the method comprising the step of incubating the sample in an enrichment medium for a period of less than 30 minutes prior to combining the sample with the solution to produce the sample solution.

36. A method according to claim 35 further comprising the step of treating the sample in a buffer solution for weakening a cell membrane of the bacterium prior to the step of applying the force application means to the sample solution.

37. A method of detecting the presence and quantity of an analyte in a sample comprising the following steps:
Obtaining the sample;
Incubating the sample for a period of time;
Combining the sample with a solution to produce a sample solution;
labelling the analyte with a detectable marker;
applying a measured volume of the sample solution to an assay device that is adapted to display said labelled analyte; and
detecting a number of labelled analyte units with a detecting means.

38. A method according to claim 37 wherein the detecting means is a microscope.

39. A method according to claim 37 wherein the analyte is a pathogen.

40. A method according to claim 38 wherein the analyte is elected from the group consisting of bacteria, viruses and fungi.

41. A method according to claim 38 wherein the analyte is a bacterium.

42. A method according to claim 41 wherein the detectable marker is a fluorescent dye.

43. A method according to claim 37 further comprising the steps of counting the number of the analyte units detected and calculating a concentration of analyte units in the measured volume of the sample solution.

44. A method according to claim 43 wherein the detecting means further includes a computer coupled to the microscope for calculating the quantity of analyte present in the sample.

45. A method according to claim 37 wherein the step of combining the sample solution with the reagent is carried out in a vessel containing the reagent.

46. A method according to claim 45 wherein the vessel further contains a concentrating material.

47. A method according to claim 45 wherein the vessel is a syringe applicator.

48. A method according to claim 45 wherein the reagent is antibodies that bind specifically to the analyte.

49. A method according to claim 48 wherein the antibodies are lyophilized antibodies that are adapted to re-hydrate instantaneously upon contact with a fluid.

50. A method according to claim 45 wherein the detectable marker is a fluorescent dye.
